(19) United States
(12) Patent Application Publication
(10) Pub. No.: US 2022/0133425 A1
(43) Pub. Date: May 5, 2022

(54) UNIVERSAL WATER-PROOF DISINFECTION BOX FOR INTERVENTIONAL ROBOT

(71) Applicant: BEIJING WEMED MEDICAL EQUIPMENT CO., LTD., Beijing (CN)

(72) Inventor: Tao Huang, Beijing (CN)

(73) Assignee: BEIJING WEMED MEDICAL EQUIPMENT CO., LTD.

(21) Appl. No.: 17/210,728

(22) Filed: Mar. 24, 2021

Related U.S. Application Data
(63) Continuation of application No. PCT/CN2021/073710, filed on Jan. 26, 2021.

(30) Foreign Application Priority Data
Oct. 29, 2020 (CN) 202011181303X

(51) Int. Cl. A61B 50/30; A61B 34/30; A61B 17/00; B25J 9/10

(52) U.S. Cl. CPC A61B 50/30; A61B 34/30; A61B 17/00234; A61B 2050/3014; A61B 2017/00889; A61B 2017/00292; A61B 2034/301; B25J 9/102

(57) ABSTRACT

A universal water-proof disinfection box for interventional robot is disclosed. The box includes a sterile box body, a sterile box cover, a catheter drive assembly and the guide wire drive assembly. One end of the sterile box body is provided with a Y-valve assembly. One end of the Y-valve assembly is rotated on one end of the sterile box body, and the other end is magnetically connected with the sterile box body. The middle part of the sterile is provided with an engagement through hole. The bottom of the axle shaft of the Y-valve driving gear rotate in the shaft hole, and the bottom of the axle have the axle wheel that matched with a motor output gear in the advancement mechanism.

### CROSS-REFERENCE TO RELATED APPLICATIONS

This application is a continuation of International Patent Application No. PCT/CN2021/073710 with a filing date of Jan. 26, 2021, designating the United States, now pending, and further claims priority to Chinese Patent Application No. 202011181303.X with a filing date of Oct. 29, 2020. The content of the aforementioned applications, including any intervening amendments thereto, are incorporated herein by reference.

### TECHNICAL FIELD

This disclosure relates generally to minimally invasive vascular interventional surgery, in particular to a control technology for the end-to-end control of a robot in an interventional operation to ensure a sterile environment in the operation. More specifically, the disclosure relates to a disposable sterile disinfection box matched with a robot, which is at the same time suitable for angiography operation and treatment operation.

### BACKGROUND

Minimally invasive interventional therapy for cardiovascular and cerebrovascular diseases is the main treatment for cardiovascular and cerebrovascular diseases. Compared with traditional surgery, it has obvious advantages such as small incision and short postoperative recovery time. Cardio-cerebrovascular intervention is a process in which the doctor manually delivers catheters, guide wires, stents and other instruments into the patient's body to complete the treatment.

Interventional radiology has the following two problems: first, during the operation process, as Digital subtraction angiography (DSA) emits X-rays, the physical strength of the doctor decreases quickly, and the attention and stability of the doctor decrease, there'll be a decrease for the operation precision. Due to the improper pushing force, vascular intima damage, vascular perforation and other accidents easily occur, which will lead to the danger of the patient's life. Second, the cumulative effects of long-term ionizing radiation can dramatically increase doctors' chances of developing leukemia, cancer, and acute cataracts. The phenomenon that doctors accumulate rays continuously because of interventional operation has become a problem that can not be ignored, which damages the professional life of doctors and restricts the development of the interventional operation.

Interventional radiology is the basis of diagnosing cardiovascular and cerebrovascular diseases, and is also a necessary step for further treatment. By using the robot technology to effectively cope with this problem, and the accuracy and stability of the operation can be greatly improved. At the same time, the injury of the interventional doctor caused by the radiation can be effectively reduced, so as to reduce the occurrence rate of accidents during the operation. Therefore, more and more attention has been paid to the cardiovascular and cerebrovascular surgery interventional robot, which has gradually become the key research object in the field of medical robot in the advanced countries of science and technology.

However, interventional operation needs to be carried out in a sterile environment, and following problems exist for the sterile environment of the interventional operation robot in China:

(1) disinfection of robots is cumbersome and does not meet the actual needs of surgery;

(2) the structure of robots is relatively bulky and complex, the size of which is large, the installation and the use of it is inconvenient;

(3) during the operation of the robots, the installation of the guide wire and the catheter is inconvenient, and the catheter and guide wire cannot be easily replaced during the operation;

(4) the guide wires cannot be pushed and rotated at the same time;

(5) the blood is easy to drip into the robot during the operation;

(6) the device is easy to slip during the advancement of the guide wire, which affects the operation effect;

(7) cost of consumables in operation is relatively high, which is not conducive to popularization;

(8) there being no universal disinfection box suitable for interventional radiology and therapeutic operation at the same time.

In the patent CN211355867U, a disposable sterile disinfection box for an interventional surgical robot is disclosed, which solves some of the above-mentioned problems, but there are still some problems existing in water-proof and the universality of interventional radiology in practice.

Therefore, providing a universal water-proof disinfection box for interventional robot is an urgent problem to be solved by those skilled in the art.

### SUMMARY

The present disclosure aims to solve at least one of the above-mentioned technical problems existing in the prior art to some extent.

Interventional radiology is the basis of diagnosing cardiovascular and cerebrovascular diseases and the precondition of further treatment, and the procedure of treatment is the necessary step to relieve the pain. Different from the interventional procedure, the realization process of the interventional radiology is different. The interventional surgical sterilization box disclosed in the prior art can not be used for the interventional radiology, because the angiography operation requires the rotational control of the angiography catheter, so as to be smoothly delivered to the coronary ostium, and achieve the purpose of angiography. However, the existing interventional sterilization box can not realize the rotation of the angiography catheter.

Therefore, an object of the present disclosure is to provide a universal water-proof disinfection box for interventional robot, which solves the technical problem that the interventional radiology and therapeutic operation sterilization box cannot be universally used.

The disclosure provides a universal water-proof disinfection box for interventional robot. The universal water-proof disinfection box includes a sterile box body, a sterile cover hinged to one side of the sterile box body. A catheter drive assembly and a guide wire drive assembly are fixed on the sterile box body, and one end of the sterile box body is provided with a Y-valve assembly.

The Y-valve assembly includes a Y-valve fixing member, a Y-valve holding member, a Y-valve main body and Y-valve driving gear.

One end of the Y-valve fixing member is rotated on one end of the sterile box body in an advancing direction of a catheter and a guide wire. Another end of the Y-valve fixing member is magnetically connected with the sterile box body. An engagement through hole is provided in the middle of the Y-valve fixing member. A shaft hole at a position corresponding to the engagement through hole is provided on the sterile box body. A bottom axle of the Y-valve drive gear is rotated in the shaft hole and the bottom of the axle is provided with an axle gear engaged with a motor output gear in a propulsion mechanism, The Y-valve drive gear is provided in the engagement through hole. The Y-valve holding member includes at least two sets of arc-shaped members connectable as a ring. A toothed ring meshed with Y-valve drive gear is provided on the ring. One end of the Y-valve main body is fixed in Y-valve holding member through an elastic filler, and another end of the Y-valve main body is fixed on the Y-valve fixing member.

According to the above technical solution, compared with the prior art, the present disclosure discloses a universal water-proof disinfection box for interventional robot with a changed structure of the Y-valve assembly thereof. Specially:

first, one end of Y-valve main body is fixed in the Y-valve holding member through an elastic filter. Through the deformation of the elastic filler, different specifications of the Y valve body can be used, so that different specification of the catheter or contrast catheter can be installed.

Second, the Y-valve driving gear is driven by the motor output gear in the propulsion mechanism, and the toothed ring on the Y-valve holding member can be driven at the same time, thus a universality of the interventional radiology and therapeutic operation disinfection box is realized.

Third, one end of a Y-valve fixing member is rotated on one side of the sterile box body in an advancing direction along a catheter and a guide wire, and the other end of the Y-valve fixing member is magnetically connected with the sterile box body, which allows the physician to replace the guide wire and catheter and fix the Y-valve main body.

Further, the Y-valve fixing member includes a fixing plate, an engaging ring body, a hinge and a claw. The fixing plate is bar shaped. A bottom of one end of the fixing plate is magnetically connected with the sterile box body, The other end of the fixing plate is integrally connected with one end of the engaging ring body. The engagement through hole is formed in the middle of the engaging ring body. The other end of the engagement ring body is connected with the hinge. The hinge is hinged with a hinge block provided on the sterile box body near an outside of the shaft hole. At least two sets of the claw are sequentially arranged along a longitudinal direction of the fixing plate. The other end of the Y-valve main body is engaged with the claw. The claw has certain elasticity, and an opening is formed at the top of the claw, which is convenient for installation and disassembly.

Further, a first Y-valve electromagnet near an inner side of the shaft hole is provided on the sterile box body. The first Y-valve electromagnet is magnetically connected with a second Y-valve electromagnet corresponding to a bottom position of the fixing plate, which thereby facilitating the fixation of the Y-valve main body.

As the moving block in the existing sterilization box adopts the design of a square block, in the practical operation, the waterproof membrane (isolation film) can easily block the iron piece fixed at the back of moving block, which will affect the connection of the electromagnet to the moving block.

Therefore, it is a second object of the present disclosure to provide a sterile box which does not interfere with the motion of the moving block.

A driving block base is formed from extending a driving block backward corresponding with the guide wire drive assembly. An area of the driving block base is smaller than the area of an driving block iron piece. The driving block iron piece is embedded in a driving block through slot of the sterile box body and is in contact with a driving block isolation film. A driven block base is formed from extending a bottom of a driven block corresponding to the guide wire drive assembly downwardly. The area of a driven block base is small than the area of the bottom of a driven block iron piece. The driven block iron piece is embedded in a driven block through slot of the sterile box body and is in contact with a driven block isolation film. Thus, the moving block of this sterilization box changes the shape of the moving block, such that the iron pieces of the moving block are partially embedded in the through slot of the sterilization box, and thus the isolation film does not interfere with the matching action of the iron piece and the electromagnet on the moving block.

In prior art, the waterproof film is adhered to the sterilization box body, thus the waterproof film is difficult to install and is easy to fall off after installation, and the waterproof property is not reliable.

For this reason, it is a third object of the present disclosure to provide a sterilization box having good waterproof property.

The driving block isolation film is clamped on the driving block through slot through fixing a first active clamp frame and a second active clamp frame thereon. The driven block isolation film is clamped by the driven block through slot through fixing a first driven clamp frame and a second driven clamp frame thereon. Therefore, with the method of fixing the two clamp frames by screws, the isolation film is tightly fixed, which is not only convenient for installation, but also more reliable in waterproof and antifouling effects.

Further, an elongated hole is provided on a side of the sterile box body near the driven block through slot for sliding the driven friction wheel axle of the catheter drive assembly. A top of the elongated hole is provided with a driven friction wheel bracket. The driven friction wheels axle is provided in the driven friction wheel bracket. An installation slot is provided at a bottom position of the elongated hole corresponding to the sterile box body. A bottom fixing piece is embedded in the installation slot. A middle part of a guide tube isolation film protrudes upward and enters the drive hole at the bottom of the driven friction wheel axle through a bottom insertion hole of the driven friction wheel bracket and then fixed on the bottom fixing piece through the elongated hole in a downward direction. The drive hole at the bottom of the robot fork piece and the axle of the driven friction wheel are separated with the elongated hole through the guide tube isolation film, so that the waterproof and antifouling performance of the sterilization box is further improved. In addition, the bottom of the guide tube isolation film is fixed in the installation slot by a bottom fixing plate, and the fixing is reliable.

Further, a detection rod through-hole of a robot detection rod is provided on an other side of the sterile box body close to the driven block through slot. A probe rod isolation film is fixed on both sides of the detection rod through hole through a first probe rod clam frame and a second probe rod clamp frame respectively. A space for the robot detection rod to move up and down is formed from upwardly raising a top of the probe rod isolation film through the detection rod through hole Therefore, an isolation film raised upward is arranged at the robot detection rod and the detection through hole of the sterilization box body, so that all the holes on the sterilization box body are completely closed, and the waterproof and antifouling performance is increased.

### BRIEF DESCRIPTION OF THE DRAWINGS

In order to illustrate the embodiments of the present disclosure or the technical solution in the prior art more clearly, a brief description of the embodiments or the drawings to be used in the description will be given below. And it is obvious that, the drawings in the following description are merely embodiments of the present disclosure, and other drawings may also be obtained from the drawings provided without any creative effort by those skilled in the art.

FIG. 1 is a structural schematic diagram of the universal water-proof disinfection box for interventional robot by the present disclosure;

FIG. 2 and FIG. 3 are exploded views of the Y-valve assembly of the universal water-proof disinfection box for interventional robot by the present disclosure;

FIG. 4 is an enlarged view of the Y-valve assembly;

FIG. 5 is a view showing a structure of the Y-valve fixing member;

FIG. 6 and FIG. 7 are schematic diagrams showing the structure of the driving block and the driven block in the guide wire drive assembly;

FIG. 8 is a schematic view showing a part of the sterile box body;

FIG. 9 is an exploded view of the driven friction wheel axle, driven friction wheel bracket, the guide tube isolation film, and the guide tube isolation film without showing the sterile box body;

FIG. 10 is an exploded view of the robot detection rod, the first probe rod clam frame, the second first probe rod clam frame and the first probe rod clam frame;

In the drawings:

101—the sterile box body, 1011—the shaft hole, 1012—the hinge block, 1013—the first Y-valve electromagnet, 1014—the driving block through slot, 1015—the driven block through slot, 1016—the second active clamp frame, 1017—the second driven clamp frame, 102—the sterile box cover, 103—the catheter drive assembly, 1031—the driven friction wheel axle, 1032—the driven friction wheel bracket, 1033—the bottom fixing piece, 1034—the guide tube isolation film, 104—the guide wire drive assembly, 1041—the driving block, 10411—the driving block base, 10412—the driving block iron piece, 1042—the driven block, 10421—the driven block base, 10422—the driven block iron piece, 105—the Y-valve assembly, 1051—the Y-valve fixing member, 10511—the engagement through hole, 10512—the fixing plate, 10513—the engaging ring body, 10514—the claw, 10515—the hinge, 1052—the Y-valve holding member, 10521—the toothed ring, 1053—the Y-valve main body, 1054—the Y-valve drive gear, 106—the robot detection rod, 1061—the detection rod through-hole, 1062—the first probe rod clam frame, 1063—the second probe rod clam frame, 1064—the probe rod isolation film, 107—the motor output gear.

### DETAILED DESCRIPTION OF EMBODIMENTS

Hereinafter, embodiments of the present disclosure will be described in detail, embodiments of which are shown in the accompanying drawings, in which the same or similar elements or elements having the similar or similar functions are denoted by the same reference numerals throughout. The embodiments described below by reference to the accompanying drawings are exemplary and intended to explain the disclosure and should not be construed as limiting the disclosure.

In the description of the disclosure, it is to be understood that, the terms “up”, “down”, “front”, “back”, “left”, “right”, “vertical”, “horizontal”, “top”, “bottom”, “inside”, “outside” etc., the orientation or positional relationship indicated is based on the shown in the drawings, merely to facilitate the description of the disclosure and to simplify the description, rather than indicating or implying that the devices or elements referred to must have a particular orientation, be constructed and operate in a specific orientation, and therefore it should not be construed as limiting the disclosure.

In addition, the term “first,” “second” are used for descriptive purpose only and are not to be construed as indicating or implying relative importance or implicitly indicate the number of technical features indicated. Thus, a feature defined as “first” or “second” may include one or more of the features, either explicitly or implicitly. In the description of the present disclosure, “plural” means two or more than two, unless otherwise specifically defined.

In the present disclosure, unless otherwise expressly specified and defined, the terms “install,” “connect,” and “fix” are to be understood in a broad sense. For example, a fixed connection or a detachable connection, or in one piece; either mechanically or electrically connected; either directly or indirectly connected through an intermediate medium, either in communication between the two elements or in an interactive relationship between them. The specific meanings of the above terms in the present disclosure may be understood by those of ordinary skill in the art as the case may be.

In the present disclosure, unless otherwise expressly specified and defined, the first feature is “up” or “down” to the second feature may comprise the first and second features in direct contact; it is also possible to include the first and second features not in direct contact but by means of a further feature contact between them. In addition, that word “up”, “above” and “on” of the first feature include the first feature being directly above and obliquely above the second feature, or simply indicate that the level of the first feature is higher than that of the second feature. If the first feature is “down”, “below” and “under” the second feature includes the first feature being directly below and diagonally below the second feature, or simply indicating that the height of the first feature is less than the second feature.

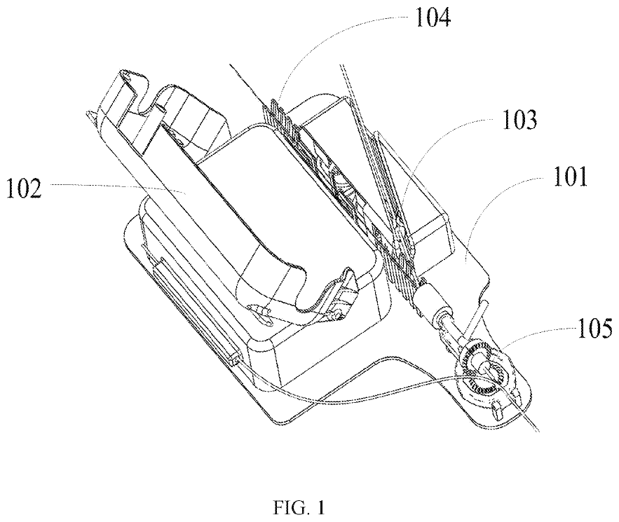

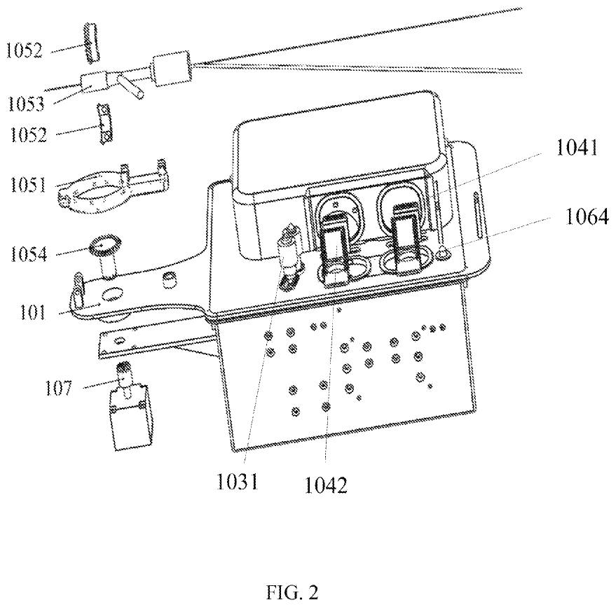

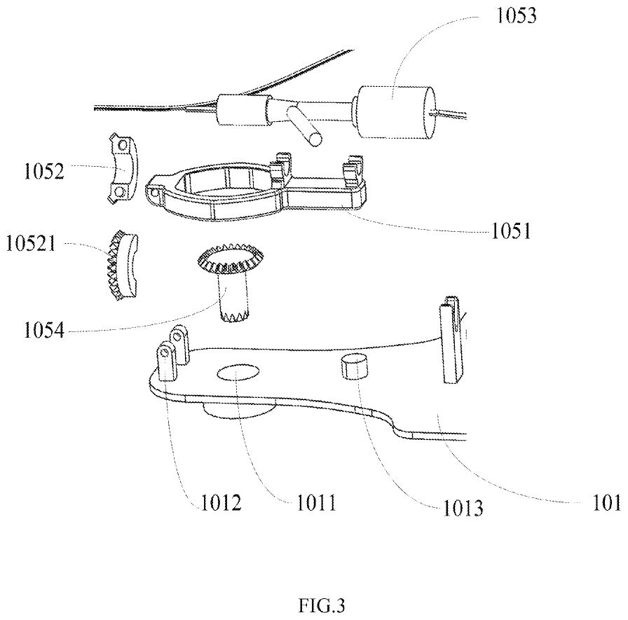

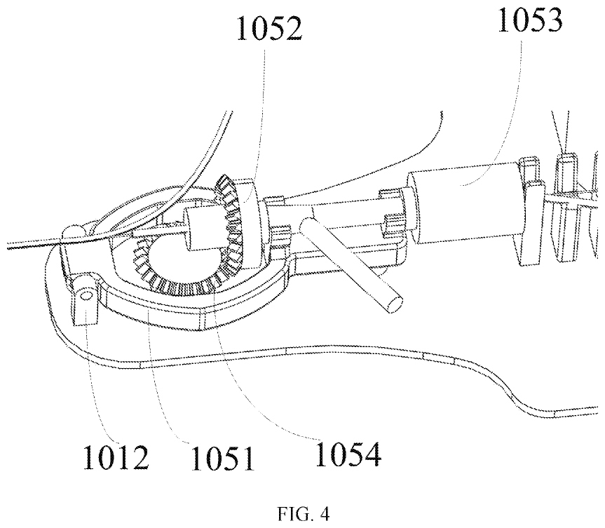

Referring to FIGS. 1-4, in one embodiment of the present disclosure, a universal water-proof disinfection box for interventional robot is disclosed. The water-proof disinfection box includes a sterile box body 101 and a sterile box cover 102 hinged to one side of the sterile box body 101. A catheter drive assembly 103 and a guide wire drive assembly 104 are fixed on the sterile box body 101. A Y-valve assembly 105 is provided on one end of the sterile box body 101.

The Y-valve assembly 105 includes a Y-valve fixing member 1051, a Y-valve holding member 1052, a Y-valve main body 1053 and a Y-valve drive gear 1054.

One end of the Y-valve fixing member 1051 is rotated on one end of the sterile box body 101 in an advancing direction of a catheter and a guide wire. Another end of the Y-valve fixing member 1051 is magnetically connected with the sterile box body 101. An engagement through hole 10511 is provided in the middle of the Y-valve fixing member 1051. A shaft hole 1011 at a position corresponding to the engagement through hole 10511 is provided on the sterile box body 101. A bottom axle of the Y-valve drive gear 1054 is rotated in the shaft hole 1011, and the bottom of the axle is provided with an axle gear engaged with a motor output gear 107 in a propulsion mechanism; the Y-valve drive gear 1054 is provided in the engagement through hole 10511. The Y-valve holding member 1052 comprises at least two sets of arc-shaped members connectable as a ring. A toothed ring 10521 meshed with Y-valve drive gear 1054 is provided on the ring. One end of the Y-valve main body 1053 is fixed in Y-valve holding member 1052 through an elastic filler, and another end of the Y-valve main body 1053 is fixed on the Y-valve fixing member 1051.

the present disclosure discloses a universal water-proof disinfection box for interventional robot with a changed structure of the Y-valve assembly thereof. Specially:

First, one end of Y-valve main body is fixed in the Y-valve holding member through an elastic filter. Through the deformation of the elastic filler, different specifications of the Y valve body can be used, so that different specification of the catheter or contrast catheter can be installed.

Second, the Y-valve driving gear is driven by the motor output gear in the propulsion mechanism, and the toothed ring on the Y-valve holding member can be driven at the same time, thus a universality of the interventional radiology and therapeutic operation disinfection box is realized.

Third, one end of a Y-valve fixing member is rotated on one side of the sterile box body in an advancing direction along a catheter and a guide wire, and the other end of the Y-valve fixing member is magnetically connected with the sterile box body, which allows the physician to replace the guide wire and catheter and fix the Y-valve main body.

The sterile box cover can be rotated 150 degrees, with an electromagnet at the bottom for holding, and an open handle at the top. The elastic filler may be sponge, silica gel or the like.

The motor output gear drives the Y-valve drive gear, which in turn drives the toothed ring on the Y-valve holding member to further drives the angiography catheter to rotate. Forward and reverse rotation of the motor corresponds to clockwise and counter-clockwise rotation of the catheter respectively. The sterile box body is provided with a semi-closed hose, the inner diameter of the semi-closed hose is larger than the diameter of the catheter and can be sleeved on the outside of the angiography catheter, and the head of the hose is fixed on the outer sheath. When the advancement mechanism is moved as a whole, the angiography catheter can be moved along the hose into or out of the body.

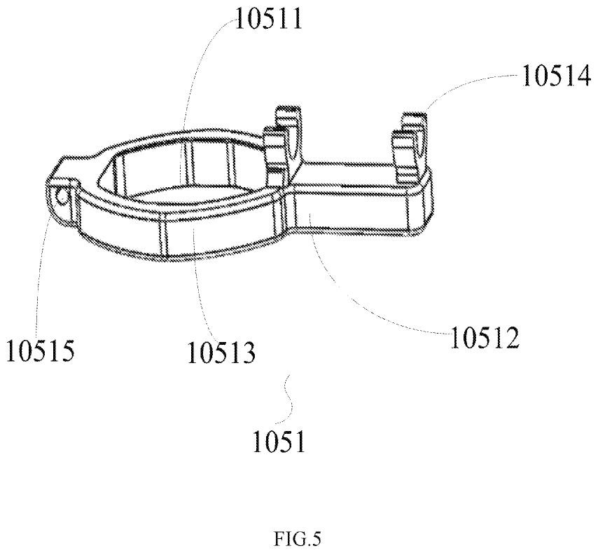

Referring to FIG. 5, the Y-valve fixing member 1051 includes a fixing plate 10512, an engaging ring body 10513, a hinge 10515 and a claw 10514. The fixing plate 10512 is bar shaped. The bottom of one end of the fixing plate 10512 is magnetically connected with the sterile box body 101. The other end of the fixing plate 10512 is integrally connected with one end of the engaging ring body 10513. The engagement through hole 10511 is formed in the middle of the engaging ring body 10513. The other end of the engagement ring body 10513 is connected with the hinge 10515. The hinge 10515 is hinged with a hinge block 1012 provided on the sterile box body 101 near an outside of the shaft hole 1011. At least two sets of the claw 10514 are sequentially arranged along a longitudinal direction of the fixing plate 10512. The other end of the Y-valve main body 1053 is engaged with the claw 10514.

A first Y-valve electromagnet 1013 near an inner side of the shaft hole 1011 is provided on the sterile box body 101. The first Y-valve electromagnet 1013 is magnetically connected with a second Y-valve electromagnet corresponding to a bottom position of the fixing plate 10512.

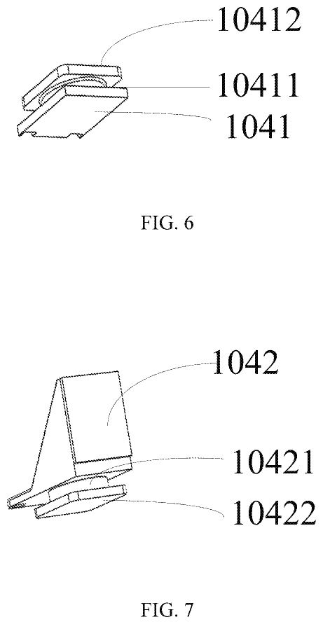

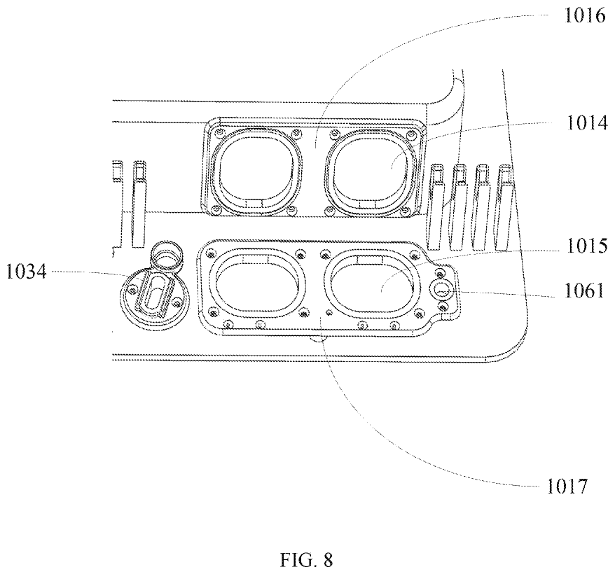

Referring to FIGS. 6 and 7, in another embodiment of the present disclosure, a driving block base 10411 is formed from extending a driving block 1041 backward corresponding with the guide wire drive assembly 104. An area of the driving block base 10411 is smaller than the area of an driving block iron piece 10412. The driving block iron piece 10412 is embedded in a driving block through slot 1014 of the sterile box body 101 and is in contact with a driving block isolation film. A driven block base 10421 is formed from extending a bottom of a driven block 1042 corresponding to the guide wire drive assembly 104 downwardly. The area of a driven block base 10421 is small than the area of the bottom of a driven block iron piece 10422. The driven block iron piece 10422 is embedded in a driven block through slot 1015 of the sterile box body 101 and is in contact with a driven block isolation film. Thus, the moving block of this sterilization box changes the shape of the moving block, such that the iron pieces of the moving block are partially embedded in the through slot of the sterilization box, and thus the isolation film does not interfere with the matching action of the iron piece and the electromagnet on the moving block.

Referring to FIG. 8, in other embodiments of the present disclosure, the driving block isolation film is clamped on the driving block through slot 1014 through fixing a first active clamp frame and a second active clamp frame 1016 thereon. The driven block isolation film is clamped by the driven block through slot 1015 through fixing a first driven clamp frame and a second driven clamp frame 1017 thereon. Therefore, with the method of fixing the two clamp frames by screws, so that the isolation film is tightly fixed, which is not only convenient for installation, but also more reliable in waterproof and antifouling effects.

Referring to FIG. 8, the driving block isolation film is clamped on the driving block through slot 1014 through fixing a first active clamp frame and a second active clamp frame 1016 thereon. The driven block isolation film is clamped by the driven block through slot 1015 through fixing a first driven clamp frame and a second driven clamp frame 1017 thereon. The waterproof and antifouling performance of the sterilization box is further improved. In addition, that bottom of the guide tube isolation film is fixed in the installation slot by a bottom fixing plate, and the fixing is reliable.

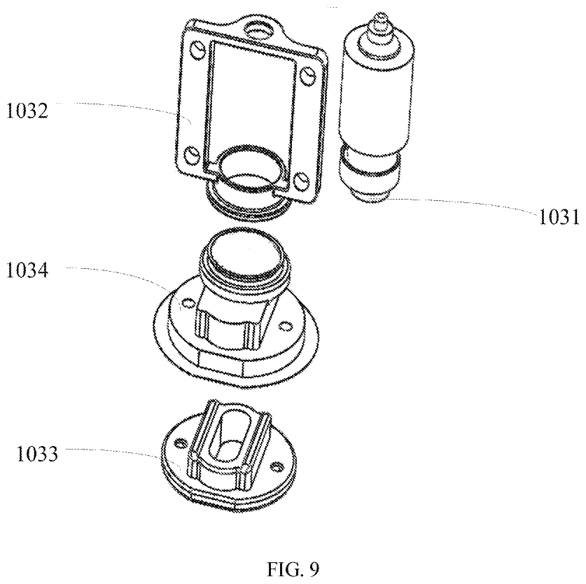

Referring to FIG. 1 and FIG. 9, an elongated hole is provided on a side of the sterile box body 101 near the driven block through slot 1015 for sliding the driven friction wheel axle 1031 of the catheter drive assembly 103. A top of the elongated hole is provided with a driven friction wheel bracket 1032. The driven friction wheels axle 1031 is provided in the driven friction wheel bracket 1032. An installation slot is provided at a bottom position of the elongated hole corresponding to the sterile box body 101. A bottom fixing piece 1033 is embedded in the installation slot. A middle part of a guide tube isolation film 1034 protrudes upward and enters the drive hole at the bottom of the driven friction wheel axle 1031 through a bottom insertion hole of the driven friction wheel bracket 1032 and then fixed on the bottom fixing piece 1033 through the elongated hole in a downward direction. The drive hole at the bottom of the axle of the driven friction wheel and the long holes are separated by the guide tube isolation film, so that the waterproof and antifouling performance of the sterilization box is further improved. In addition, that bottom of the guide tube isolation diaphragm is fixed in the mounting groove by a bottom fix plate, and the fixing is reliable.

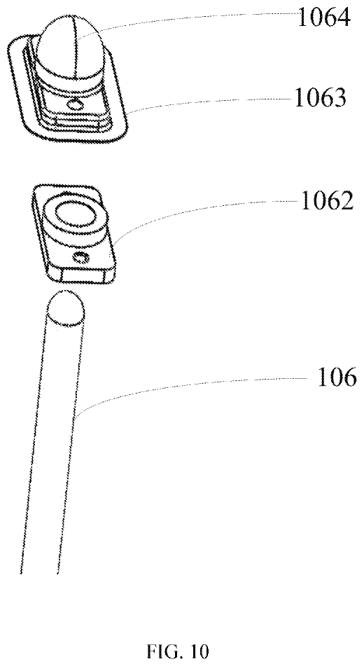

More advantageously, referring to FIG. 10, a detection rod through-hole 1061 of a robot detection rod 106 is provided on an other side of the sterile box body 101 close to the driven block through slot 1015. A probe rod isolation film 1064 is fixed on both sides of the detection rod through hole 1061 through a first probe rod clam frame 1062 and a second probe rod clamp frame respectively. A space for the robot detection rod 106 to move up and down is formed from upwardly raising a top of the probe rod isolation film 1064 through the detection rod through hole 1061. Therefore, an isolation film raised upward is arranged at the robot detection rod and the detection through hole of the sterilization box body, so that all the holes on the sterilization box body are completely closed, and the waterproof and antifouling performance is increased.

In the disclosure, the isolation films can be latex, and a thickness of about 0.1 mm can meet the use requirement.

The present disclosure can be used as a consumable material of an interventional surgical robot, and can also be used in conjunction with an advancement mechanism of the interventional surgical robot, for creating a sterile environment for the interventional surgical robot. The forward, backward, and rotation control of the catheter and the guide wire in the interventional surgical robot can be realized, and the surgeon can control device through the interventional surgical robot outside of the surgical room, and can control the catheter drive assembly and the guide wire drive assembly through controlling the interventional surgical robot, so as to push the catheter and the guide wire into the patient and achieve the purpose of interventional operation treatment. The sterilization box is disposable and is sterile processed before using, which guarantees the surgery sterile environment. It can be used for both interventional radiolog and interventional treatment operation. It has the advantages of simple structure, small volume, light weight, convenient loading and unloading, low cost and the like.

The sterile box and the robot advancement mechanism are connected in a plug-in type, that is, the sterile box is directly buckled on the advancement mechanism to complete the installation process without tools, and the entire processes only takes 3 seconds. After the operation, it is only need to pull out the sterile box, which is quick and convenient, and then collect them in a unified manner. The operation process is to place the guide wire or catheter, close the sterile box cover, trigger the detection rod, the robot will automatically clamp the catheter or guide wire, and then control the movement of the robot to control the movement of the catheter or the guide wire to complete the operation. The sterile box is completely waterproof, so there is no need to worry about blood falling into the robot.

In the description of the present specification, reference to the description of the terms “one embodiment”, “some embodiments”, “an example”, “a specific example”, or “some examples” or the like, is intended to refer to specific features, structures, materials or features that are included in at least one embodiment or example of the disclosure. In the specification, the schematic representations of the above terms are not necessarily directed to the same embodiments or examples. Moreover, the particular features, structures, materials, or features described may be combined in any one or more embodiments or examples in a suitable manner. In addition, those skilled in the art may join and combine the different embodiments or examples described in this specification.

Although the embodiments of the present disclosure have been shown and described above, it is to be understood that the embodiments described above are exemplary and not to be construed as limiting the disclosure. Variations, modifications, substitutions, and variations of the above-described embodiments may be made by one of ordinary skill in the art within the scope of the present disclosure.

What is claimed is:

1. A universal water-proof disinfection box for interventional robot, comprising a sterile box body (101) and a sterile box cover (102) hinged to one side of the sterile box body (101);
wherein a catheter drive assembly (103) and a guide wire drive assembly (104) are fixed on the sterile box body (101); a Y-valve assembly (105) is provided on one end of the sterile box body (101);
the Y-valve assembly (105) comprises a Y-valve fixing member (1051), a Y-valve holding member (1052), a Y-valve main body (1053) and a Y-valve drive gear (1054);
one end of the Y-valve fixing member (1051) is rotated on one end of the sterile box body (101) in an advancing direction of a catheter and a guide wire; an other end of the Y-valve fixing member (1051) is magnetically connected with the sterile box body (101); an engagement through hole (10511) is provided in the middle of the Y-valve fixing member (1051); a shaft hole (1011) at a position corresponding to the engagement through hole (10511) is provided on the sterile box body (101); a bottom axle of the Y-valve drive gear (1054) is rotated in the shaft hole (1011), and the bottom of the axle is provided with an axle gear engaged with a motor output gear (107) in a propulsion mechanism; the Y-valve drive gear (1054) is provided in the engagement through hole (10511); the Y-valve holding member (1052) comprises at least two sets of arc-shaped members connectable as a ring; a toothed ring (10521) meshed with Y-valve drive gear (1054) is provided on the ring; and one end of the Y-valve main body (1053) is fixed in Y-valve holding member (1052) through an elastic filler, and an other end of the Y-valve main body (1053) is fixed on the Y-valve fixing member (1051).

2. The universal water-proof disinfection box for interventional robot of claim 1, wherein the Y-valve fixing member (1051) comprises a fixing plate (10512), an engaging ring body (10513), a hinge (10515) and a claw (10514); the fixing plate (10512) is bar shaped; a bottom of one end of the fixing plate (10512) is magnetically connected with the sterile box body (101); the other end of the fixing plate (10512) is integrally connected with one end of the engaging ring body (10513); the engagement through hole (10511) is formed in the middle of the engaging ring body (10513); the other end of the engagement ring body (10513) is connected with the hinge (10515); the hinge (10515) is hinged with a hinge block (1012) provided on the sterile box body (101) near an outside of the shaft hole (1011); at least two sets of the claw (10514) are sequentially arranged along a longitudinal direction of the fixing plate (10512); and the other end of the Y-valve main body (1053) is engaged with the claw (10514).

3. The universal water-proof disinfection box for interventional robot of claim 2, wherein a first Y-valve electromagnet (1013) near an inner side of the shaft hole (1011) is provided on the sterile box body (101); the first Y-valve electromagnet (1013) is magnetically connected with a second Y-valve electromagnet corresponding to a bottom position of the fixing plate (10512).

4. The universal water-proof disinfection box for interventional robot of claim 1, wherein a driving block base (10411) is formed from extending a driving block (1041) backward corresponding with the guide wire drive assembly (104); an area of the driving block base (10411) is smaller than the area of an driving block iron piece (10412); the driving block iron piece (10412) is embedded in a driving block through slot (1014) of the sterile box body (101) and is in contact with a driving block isolation film; a driven block base (10421) is formed from extending a bottom of a driven block (1042) corresponding to the guide wire drive assembly (104) downwardly; the area of a driven block base (10421) is small than the area of the bottom of a driven block iron piece (10422); the driven block iron piece (10422) is embedded in a driven block through slot (1015) of the sterile box body (101) and is in contact with a driven block isolation film.

5. The universal water-proof disinfection box for interventional robot of claim 4, wherein the driving block isolation film is clamped on the driving block through slot (1014) through fixing a first active clamp frame and a second active clamp frame (1016) thereon; and the driven block isolation film is clamped by the driven block through slot (1015) through fixing a first driven clamp frame and a second driven clamp frame (1017) thereon.

6. The universal water-proof disinfection box for interventional robot of claim 4, wherein an elongated hole is provided on a side of the sterile box body (101) near the driven block through slot (1015) for sliding the driven friction wheel axle (1031) of the catheter drive assembly (103); a top of the elongated hole is provided with a driven friction wheel bracket (1032); the driven friction wheels axle (1031) is provided in the driven friction wheel bracket (1032); an installation slot is provided at a bottom position of the elongated hole corresponding to the sterile box body (101); a bottom fixing piece (1033) is embedded in the installation slot; and a middle part of a guide tube isolation film (1034) protrudes upward and enters the drive hole at the bottom of the driven friction wheel axle (1031) through a bottom insertion hole of the driven friction wheel bracket (1032) and then fixed on the bottom fixing piece (1033) through the elongated hole in a downward direction.

7. The universal water-proof disinfection box for interventional robot of claim 4, wherein a detection rod through-hole (1061) of a robot detection rod (106) is provided on an other side of the sterile box body (101) close to the driven block through slot (1015); a probe rod isolation film (1064) is fixed on both sides of the detection rod through hole (1061) through a first probe rod clam frame (1062) and a second probe rod clamp frame respectively; and a space for the robot detection rod (106) to move up and down is formed from upwardly raising a top of the probe rod isolation film (1064) through the detection rod through hole (1061).
